(19) United States
(12) Patent Application Publication
(10) Pub. No.: US 2025/0170316 A1
(43) Pub. Date: May 29, 2025

(54) PERITONEAL DIALYSIS FLUID COMPOSITION COMPRISING A COMPLEMENT INHIBITOR

(71) Applicant: INVIZIUS LIMITED, Edinburgh (GB)

(72) Inventors: Andrew HERBERT, Edinburgh (GB); Elisavet Makou, Edinburgh (GB); Magnus NICOLSON, Edinburgh (GB)

(21) Appl. No.: 18/841,849

(22) PCT Filed: Feb. 28, 2023
(86) PCT No.: PCT/GB2023/050452

§ 371 Date: Aug. 27, 2024

(30) Foreign Application Priority Data
Feb. 28, 2022 (GB) 2202728.8

(51) Int. Cl. A61M 1/28; A61K 38/16; A61K 38/17

(52) U.S. Cl. CPC A61M 1/287; A61K 38/164; A61K 38/1709

(57) ABSTRACT

A composition for the use in peritoneal dialysis (PD) is hereby provided, the composition comprising a biologically compatible solvent, an osmotic agent and a complement inhibitor. Methods of manufacturing and of use of the composition are also provided.

### FIELD

The present disclosure relates to compositions for use in peritoneal dialysis, and methods of peritoneal dialysis.

### BACKGROUND

Peritoneal dialysis (PD) is one of the fastest growing treatments option for patients with kidney failure and is a better option than haemodialysis (HD), especially for patients with residual kidney function and intolerance of rapid fluid balance changes associated with haemodialysis. PD offers treatment flexibility and reduces visits to dialysis centres. Choosing PD home dialysis provides benefits such as reduced medications and fewer food restrictions. PD allows patients to remain active, pursue a normal education or maintain their work while haemodialysis (HD), which requires patients to be dialysed three times a week for at least three hours, limits the possibilities of work for patients. PD is a form of dialysis that uses the inner lining of the abdomen (peritoneum) and dialysis solution to filter blood when the kidney is not functioning effectively. Patients can use peritoneal dialysis at home without assistance. There are two forms of peritoneal dialysis. CAPD involves three to four fluid exchanges during the day and a long dwell during the night. APD or automated peritoneal dialysis is a technique where a machine called cycler operates the fluid exchanges during the night, while the patient sleeps, allowing him or her to have only one exchange during the day. A new generation of connected cyclers allows 24/7 monitoring of patients. These advantages make PD popular with kidney patients and clinicians alike and will further fuel the home care segment growth of peritoneal dialysis market.

PD accounts for the treatment of approximately 10% of the dialysis population worldwide. It is more cost effective than haemodialysis, but with similar outcomes, whilst allowing a home-based therapy. However, there are high rates of transfer of patients from PD to HD. Only about 15% of patients continue to be treated by PD after 5 years.

The major concern limiting long-term treatment with PD is the loss of the peritoneal membrane integrity and function after repeated and prolonged exposure to the dialysis solutions. Overtime on PD, the peritoneal membrane undergoes structural alterations, including progressive fibrosis, angiogenesis, and vasculopathy. These morphological changes are paralleled by the acceleration of solute transport, earlier dissipation of the osmotic gradient induced by glucose, and, ultimately, loss of ultrafiltration capacity.

Accordingly, there is a need for improved PD treatments that extend the time PD remains an effective therapy.

At least some aspects of the disclosure aim to address at least one of the above needs.

### SUMMARY

According to a first aspect there is provided a composition for the use in peritoneal dialysis (PD), the composition comprising a biologically compatible solvent, an osmotic agent and a complement inhibitor.

The biologically compatible solvent may be water and the composition may therefore be an aqueous composition.

The osmotic agent may comprise a soluble carbohydrate. The soluble carbohydrate may be a monosaccharide, an oligosaccharide, or a polysaccharide. In some embodiments, the soluble carbohydrate may be a sugar or a sugar derivative.

The osmotic agent may comprise amino acids. For example, the osmotic agent may comprise one or more of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, L-carnitine or other suitable non-natural amino acids and/or derivatives and/or mixtures and/or polymers thereof.

Accordingly, the osmotic agent may comprise proteins. For example, the osmotic agent may comprise one or more proteins such as human serum albumin or gelatin.

The composition may comprise one or more stabilisers. The one or more stabilisers may be sodium octanoate or N-acetyl-L-tryptophanate, for example.

As used herein, the term “osmotic agent” refers to any soluble agent that increases the osmotic potential of the composition such that the composition is hypertonic or at least isotonic when retained within the peritoneal cavity such that toxins are drawn across the peritoneal membrane into the composition.

Typical compositions used in peritoneal dialysis (PD) are aqueous solutions comprising at least one osmotic agent. The composition is used in PD by transferring the composition into the peritoneal cavity of a patient and allowing it to remain there for a sufficient length of time to draw toxins across the peritoneal membrane from the blood of the patient into the composition. The composition is then subsequently removed from the peritoneal cavity, thereby removing the toxins from the patient's body.

During prolonged PD treatment where a patient uses repeated PD treatment (e.g. multiple times a day, daily, or weekly) over a period of time the peritoneal membrane undergoes structural alterations, including progressive fibrosis, angiogenesis, and vasculopathy. The mesothelial cells that form the monolayer that lines the peritoneal membrane have a distinct cobblestone-like morphology and properties such as regulation of peritoneal permeability. These morphological changes the cells undergo are paralleled by the acceleration of solute transport, earlier dissipation of the osmotic gradient induced by glucose (a commonly used osmotic agent), and, ultimately, loss of ultrafiltration capacity.

The underlying process is a transformation of mesothelial cells form an endothelial to a mesenchymal phenotype, initiated by the disruption of intercellular junctions and loss of polarity resulting in acquisition of fibrogenic features. In addition, treatment of mesothelial cells with high-glucose PD solutions induces expression of transforming growth factor B. This ultimately results in the increased expression of some markers corresponding to the mesenchymal phenotype, such as Vimentin, N-Cadherin, VEGF-A, α-SMA, IL-17A and a marked down-regulation of endothelial markers such as E-cadherin.

The biological origin for these structural alterations is unclear. It has been recently suggested (Borceux et al. Peritoneal Dialysis International 2020, Vol. 40(2) pgs. 115-123) that complement system activation is at least partially involved in these peritoneal membrane alterations.

The complement system is a set of 40-50 proteins in the blood whose primary function is to provide a first line of defence against infection, but also plays an important role in the clearance of necrotic, apoptotic, or damaged cells and immune complexes. An improperly regulated complement system can damage host or self-cells as well as bacterial ones. Borceux et al. (full reference above) states that molecules belonging to the complement system such as C3-C9, and Factors B, D and H are present in PD effluent (i.e. the PD fluid that is removed from the patient at the end of a PD treatment). These data may indicate either a clearance of those molecules from the circulation by drawing them through the peritoneal membrane from the patient's blood, a local intraperitoneal production of these proteins, or a combination of both phenomena.

In addition, Bartosova et al. (Bartosova et al. J Am Soc Nephrol 2018, Vol 29(1) pgs 268-282) recently demonstrated complement system activation and increased deposition of proteins from the classical and alternative pathway (e.g., C1q, C3d, and C5b-9) in peritoneal parietal and omental arterioles, from children undergoing PD and treated with low GDPs solutions in comparison to end-stage renal disease and healthy controls. Furthermore, the complement activation induced by the PD solutions is closely related to with the degree of vascular disease as is the TGF-β activation.

The inventors have found that the use of a complement inhibitor in a PD fluid, such as the composition of the present aspect, at least reduces or slows the rate at which the peritoneal membrane undergoes at least one of the structural or functional alterations described above. Accordingly, patient's undergoing PD treatments may be able to continue using PD treatment for longer periods of time before the peritoneal membrane becomes compromised.

In embodiments where the osmotic agent is a soluble carbohydrate, the soluble carbohydrate may be selected from the group consisting of: glucose, dextrose (L-glucose), fructose, galactose, maltose, xylitol, mannitol, sorbitol, maltodextrin, icodextrin, sucrose, hyaluronic acid, or derivations or fragments or mixtures thereof. The soluble carbohydrate may be selected from the group consisting of: glucose, dextrose (L-glucose), maltodextrin or icodextrin or derivatives or fragments or mixtures thereof.

The term “derivative” refers to a biological molecule that has been altered chemically or genetically in a way which does not significantly reduce its biological activity. A derivative of a biological molecule may improve the biological activity of the biological molecule. A derivative may be a functional derivative or a biologically effective analogue of the parent biomolecule.

The complement inhibitor may inhibit complement activation or may accelerate de-activation of complement or may inhibit the central amplification loop or it may inhibit the downstream effector functions. For example, the complement inhibitor may bind to a component of a complement pathway and directly inhibit activation of the complement pathway or bind to a component of a complement pathway and accelerate de-activation of the complement pathway.

The complement inhibitor may be a small molecule, a peptide, a macrocyclic peptide, a monoclonal antibody, another recombinant protein, a native protein, an oligonucleotide, a hexaBody, an affibody, a minibody, a nanobody, a Fab, or equivalent antibody derivative, a biologic or an aptamer configured to bind to and inhibit a component of any complement pathway. The complement inhibitor may be a monoclonal antibody, another recombinant protein or an aptamer configured to bind to and activate or enhance one of the natural complement regulators. The complement inhibitor may be unmodified or modified for example with polyethylene glycol, proline-alanine/serine-rich sequences or lipids.

The complement inhibitor may be an inhibitor of Factor D, Factor B, properdin, MASPs-1 to 3, C1, C3, C3a, C3b, C4b, C5, C5a, C5b, C5aR1, C6, or membrane attack complex (MAC). The complement inhibitor may be a direct inhibitor of Factor D, Factor B, properdin, MASPs-1 to 3, C1, C3, C3a, C3b, C4b, C5, C5a, C5b C5aR1, C6, or MAC. The complement inhibitor may be an indirect inhibitor of Factor D, Factor B, properdin, MASPs-1 to 3, C1, C3, C3a, C3b, C4b, C5, C5a, C5b C5aR1, C6, or MAC.

The complement inhibitor may be an activator or enhancer of the activity of Factor H, C4bp, CR1, DAF, or MCP.

The complement inhibitor may be C1-inhibitor, also known as C1-inh, and C1 esterase inhibitor.

The complement inhibitor may inhibit MAC. The complement inhibitor may be CD59 glycoprotein, also known as MAC-inhibitory protein (MAC-IP), membrane inhibitor of reactive lysis (MIRL), or protectin.

The complement inhibitor may accelerate the decay of the C3 convertase. For example, the complement inhibitor may be selected from DAF, Factor H, CR1, VCP, or SPICE.

Methods for assessing decay accelerating activity include those described in Biggs et al. (Invest Ophthalmol Vis Sci. 2022 November; 63(12): 30, Published online 2022 Nov. 29. doi: 10.1167/iovs.63.12.30, PMID: 36445700, An Evaluation of the Complement-Regulating Activities of Human Complement Factor H (FH) Variants Associated With Age-Related Macular Degeneration) and Herbert et al. (J Immunol. 2015 Nov. 15; 195(10): 4986-4998, Published online 2015 Oct. 12. doi: 10.4049/jimmunol. 1501388, PMID: 26459349, Complement Evasion Mediated by Enhancement of Captured Factor H: Implications for Protection of Self-Surfaces from Complement).

The complement inhibitor may be selected from the group comprising [“complement inhibitor” “example source”, “(mode of action)”]: C1-INH or C1 esterase inhibitor also known as Cetor, Berinert, or Cinryze, Sanquin/CSL Behring/Takeda Pharmaceuticals (CP/LP inhibition, other serine proteases); IFX-1 also known as CaCP29, InflaRx (Blocking binding of C5a to C5aR1); Mirococept also known as APT070, King's College London MRC (Inhibition of CP and AP C3/C5 convertases); TP10 also known as CDX-1135 or soluble complement receptor 1, Avant Immunotherapeutics (Inhibition of CP and AP C3/C5 convertases); Eculizumab also known as Soliris, Alexion (Blockage of C5 activation); AMY-101, Amyndas (Inhibition of C3 activation); Ravulizumab also known as ALX1210 or Ultomiris, Alexion (Blockage of C5 activation (targets same epitope as eculizumab)); Crovalimab also known as SKY59 or RO7112689, Hoffmann-La Roche (Blockage of C5 activation (different C5 epitope)); Tesidolumab also known as LFG316, Novartis (Blockage of C5 activation (different C5 epitope); Pozelimab also known as REGN3918, Regeneron (Blockage of C5 activation (different C5 epitope)); ABP959, Amgen (Biosimilar of eculizumab); SB12, Samsung Bioepis (Biosimilar of eculizumab); Nomacopan also known as rVA576 or Coversin or OmCI, Akari Therapeutics (Inhibition of C5 activation); Zilucoplan also known as RA101495, Ra Pharmaceuticals (ALLosteric inhibition of C5 activation); Cemdisiran also known as ALN-CC5, Alnylam (Inhibition of hepatic expression of C5); APL-2 also known as Pegcetacoplan, Apellis (Inhibition of C3 activation); LNP023, Novartis (Inhibition of AP C3 convertase); Danicopan also known as ACH-4471 or ACH-0144471, Achillion (Inhibition of AP C3 convertase); Sutimlimab also known as BIV009 or TNT009, Sanofi (CP inhibition/inhibition of C1s protease); Avacopan also known as CCX168, ChemoCentryx (Antagonist of the C5aR1 receptor); Narsoplimab also known as OMS721, Omeros (LP inhibition, blockage of MASP2 activity); Zimura also known as avacincaptad pegol or ARG 1905, IVERIC Bio (Inhibition of C5 expression); Lampalizumab, Roche (Blockage of AP C3 convertase formation); CLG561 also known as NOV-7, Novartis (Properdin); IONIS-FB-LRx, Roche (Inhibition of hepatic FB expression); IPH5401, Innate Pharma (Blockade of C5aR1 signalling); GEN1029, GenMab (Enhancement of CDC against DR5+ tumours); Ruconest, Pharming (C1r/s; MASPs; C1 esterase inhibitor); ACH-5228, Achillion (FD inhibitor); ACH-5448, Achillion (FD inhibitor); APL-9, Apellis (C3 inhibitor); AAVCAGsCD59 also known as HMR59, Hemera Biosciences (expression of soluble CD59); ANX005, Annexon (C1q); ANX007, Annexon (C1q); BIVV020, Bioverativ (C1s); OMS906, Omeros (MASP3); PRO-02, Broteio (C2); AMY-103, Amyndas (C3); 5C6 also known as Compsorbin, Amyndas (FH); anti-FH.07, Sanquin, (FH); AMY-201 also known as miniFH, Amyndas (Convertases); variant mini FH (SEQ ID NO:9); SOBI005, Sobi (C5); ISU305, ISU ABXIS (C5); Mubodima, Adienne (C5); IFX-2, InflaRx (C5a); IFX-3, InflaRx (C5a); ALS-205, Alsonex (C5aR1); DF2593A, Dompé (C5aR1); Regenemab, Regenesance (C6); C6-LNA, Regenesance (C6) or PspC or functional variant or fragment thereof.

The complement inhibitor may prevent the initiation and amplification of complement and may be selected from a group consisting of: C1-INH; Sutimlimab/BIV009/TNT009; Narsoplimab/OMS721; Ruconest; ANX005; ANX007; BIVV020; PRO-02.

The complement inhibitor impairs effector functions of complement and is selected from a group consisting of: IFX-1/CaCP29; Eculizumab (Soliris); Ravulizumab/ALX1210/Ultomiris; Crovalimab/SKY59/RO7112689; Tesidolumab/LFG316; Pozelimab/REGN3918; ABP959; SB12; Nomacopan/rVA576/Coversin/OmCI; Zilucoplan/RA101495; Cemdisiran/ALN-CC5; Zimura/avacincaptad pegol; Lampalizumab; CLG561; IONIS-FB-LRx; IPH5401; GEN1029; AAVCAGsCD59/HMR59; SOBI005; ISU305; Mubodima; IFX-2; IFX-3; ALS-205; DF2593A; Regenemab; or C6-LNA.

The complement inhibitor may attenuate the amplification of complement and may be selected from a group consisting of: Mirococept (APT070); TP10/CDX-1135 (soluble complement receptor 1); AMY-101; APL-2; LNP023; Danicopan/ACH-4471/ACH-0144471; Sutimlimab/BIV009/TNT009; Lampalizumab; CLG561; IONIS-FB-LRx; ACH-5228; ACH-5448; APL-9; BIVV020; OMS906; PRO-02; AMY-103; 5C6/Compsorbin; anti-FH.07; AMY-201/miniFH; variant mini FH (SEQ ID NO:9); DAF 1-4; or PspC or functional variant or fragment thereof.

Variant mini FH is an variant of miniFH and has the sequence SEQ ID NO:9:

EDCNELPPRRNTEILTGSWSDQTYPEGTQAIYKCRPGYRSLGNVIMVCR
KGEWVALNPLRKCQKRPCGHPGDTPFGTFTLTGGNVFEYGVKAVYTCNE
GYQLLGEINYRECDTDGWTNDIPICEVVKCLPVTAPENGKIVSSAMEPD
REYHFGQAVRFVCNSGYKIEGDEEMHCSDDGFWSKEKPKCVEISCKSPD
VINGSPISQKIIYKENERFQYKCNMGYEYSERGDAVCTESGWRPLPSCE
EKSCDNPYIPNGDYSPLRIKHRTGDEITYQCRNGFYPATRGNTAKCTST
GWIPAPRCTLGGGSGGGGSGGGTSCVNPPTVQNAYIVSRQMSKYPSGER
VRYQCRSPYEMFGDEEVMCLNGNWTEPPQCKDSTGKCGPPPPIDNGDIT
SFPLSVYAPASSVEYQCQNLYQLEGNKRITCRNGQWSEPPKCLHPCVIS
REIMENYNIALRWTAKQKLYSRTGESVEFVCKRGYRLSSRSHTLRTTCW
DGKLEYPTCAKR.

DAF 1-4 is recombinant protein comprising complement control protein modules 1-4 from human Decay Acceleration Factor having the sequence SEQ ID NO:12:

DCGLPPDVPNAQPALEGRTSFPEDTVITYKCEESFVKIPGEKDSVICLK
GSQWSDIEEFCQRSCEVPTRLNSASLKQPYITQNYFPVGTVVEYECRPG
YRREPSLSPKLTCLQNLKWSTAVEFCKKKSCPNPGEIRNGQIDVPGGIL
FGATISFSCNTGYKLFGSTSSFCLISGSSVQWSDPLPECREIYCPAPPQ
IDNGIIQGERDHYGYRQSVTYACNKGFTMIGEHSIYCTVNNDEGEWSGP
PPECRGK.

The complement inhibitor may be capable of binding to a natural human complement regulator thereby increasing its activity. Accordingly, the protein may be able to act as a complement inhibitor by enhancing the activity of the patient's own natural complement regulators.

The complement inhibitor may be capable of binding to complement factor H. The complement inhibitor may be a protein capable of binding to CFH, and thereby inducing increased affinity for C3d and C3b by bound CFH compared to unbound CFH. Accordingly, the protein may be able to act as a complement inhibitor of the central amplification loop at the level of the C3 convertase, and an inhibitor of the alternative complement pathway.

The complement inhibitor may be derived from a microbial protein whose function is to protect the microbe from the complement system.

The complement inhibitor may be derived from a pneumococcal surface protein. The complement inhibitor may be derived from pneumococcal surface protein C (PspC) from Streptococcus pneumoniae. Various forms of PspC are known, e.g. variants derived from different strains of S. pneumoniae. The complement inhibitor may be derived from PspC of strain D39 (NCTC no 7466) of S. pneumoniae (SEQ ID NO 4). The complement inhibitor may be derived from CbpA of strain TIGR4 (NCTC no 7465). The complement inhibitor may comprise a fragment of PspC. The fragment of PspC may comprise a portion of the N-terminal region of PspC. The efficacy of PspCN as a complement inhibitor is demonstrated in International patent application WO 2015/055991 to the University Court of the University of Edinburgh, the disclosure of which is hereby incorporated by reference.

The sequence of the complement inhibitor PspCN is ATENEGSTQAATSSNMAKTEHRKAAKQVVDEYIEKMLREIQLDRRKHTQNVALNIKLSAIKT KYLRELNVLEEKSKDELPSEIKAKLDAAFEKFKKDTLKPGEK (SEQ ID NO 1), or a functional variant or fragment thereof, which corresponds to amino acid residues 37-140 of PspC. The full-length sequence of PspC is set out in Genbank accession no. AF068646.

The sequence of an alternative complement inhibitor related to PspCN is KQVVDEYIEKMLREIQLDRRKHTQNVALNIKLSAIKTKYLRELNVLEEKSKDELPSEIKAKLDA AFEKFKKDTLKPGEK (SEQ ID NO 2) or a functional variant or fragment thereof. This sequence is amino acids 62-140 of PspC.

The sequence of a further alternative complement inhibitor related to PspCN is

(SEQ ID NO 3)
ATENEGATQVPTSSNRANESQAEQGEQPKKLDSERDKARKEVEEYVKKI
VGESYAKSTKKRHTITVALVNELNNIKNEYLNKIVESTSESQLQILMME
SRSKVDEAVSKFEKDSSSSSSSDSSTKPEASDTAKPNKPTEPGEK,
or a functional variant or fragment thereof.

The microbially derived complement inhibitor may be derived from a pox virus, including vaccina virus complement control protein (VCP), or smallpox inhibitor of complement enzymes (SPICE) or monkeypox virus inhibitor of complement enzymes (MOPICE).

Accordingly, the microbially derived complement inhibitor may be VCP and have a sequence:

(SEQ ID NO 4)
CCTIPSRPINMKFKNSVETDANANYNIGDTIEYLCLPGYRKQKMGPIYA
KCTGTGWTLFNQCIKRRCPSPRDIDNGQLDIGGVDFGSSITYSCNSGYH
LIGESKSYCELGSTGSMVWNPEAPICESVKCQSPPSISNGRHNGYEDFY
TDGSVVTYSCNSGYSLIGNSGVLCSGGEWSDPPTCQIVKCPHPTISNGY
LSSGFKRSYSYNDNVDFKCKYGYKLSGSSSSTCSPGNTWKPELPKCVR,
or a functional variant or fragment thereof.

The microbially derived complement inhibitor may be SPICE and have a sequence:

(SEQ ID NO 5)
CCTIPSRPINMKFKNSVETDANANYNIGDTIEYLCLPGYRKQKMGPIYA
KCTGTGWTLFNQCIKRRCPSPRDIDNGHLDIGGVDFGSSITYSCNSGYY
LIGEYKSYCKLGSTGSMVWNPKAPICESVKCQLPPSISNGRHNGYNDFY
TDGSVVTYSCNSGYSLIGNSGVLCSGGEWSNPPTCQIVKCPHPTILNGY
LSSGFKRSYSYNDNVDFTCKYGYKLSGSSSSTCSPGNTWQPELPKCVR,
or functional variant or fragment thereof.

The microbially derived complement inhibitor may be MOPICE and have a sequence:

(SEQ ID NO 6)
YCTIPSRPINMKFKNSVETDANANYNIGDTIEYLCLPGYRKQKMGPIYA
KCTGTGWTLFNQCIKRRCPSPRDIDNGQLDIGGVDFGSSITYSCNSGYH
LIGESKSYCELGSTGSMVWNPEAPICESVKCQSPPSISNGRHNGYEDFY
TDGSVVTYSCNSGYSLIGNSGVLCSGGEWSDPPTCQIVKCPHPTISNGK
LLAA,
or functional variant or fragment thereof.

The term “fragment” is intended to refer to a polyamino acid of at least 3, 6, 10, 15, 30, 60 contiguous amino acids of the reference sequences or any integer therebetween. Preferably the fragment is a functional fragment. A functional fragment is a fragment that at least represents the part or parts of the protein, which are essential for the protein to be able to serve to bind and activate CFH, and can fulfil this function, for example, when used alone or in a multi-subunit form. Thus, such functional fragments may be polypeptides that are functional per se, or the fragments may be functional when linked to other polypeptides, e.g. to obtain chimeric proteins. Such functional fragments are understood to fall within the scope of the present invention. Whether a fragment is functional can be determined using the various bioassays herein described.

Fragments can be produced, inter alia, by enzymatic cleavage of precursor molecules, using restriction endonucleases for the DNA and proteases for the polypeptides. Other methods include chemical synthesis of the fragments or the production of peptide fragments encoded by DNA.

As used herein, the term “protein” can be used interchangeably with “peptide” or “polypeptide”, and means at least two covalently attached alpha amino acid residues linked by a peptide bond. The term protein encompasses purified natural products, or chemical products, which may be produced partially or wholly using recombinant or synthetic techniques. The term protein may refer to a complex of more than one polypeptide, such as a dimer or other multimer, a fusion protein, a protein variant, or derivative thereof. The term also includes modified proteins, for example, a protein modified by glycosylation, acetylation, phosphorylation, pegylation, ubiquitination, and so forth. A protein may comprise amino acids not encoded by a nucleic acid codon.

Proteins having minor modifications in the sequence are equally useful, provided they are functional, and the complement inhibitor may be a protein comprising an amino acid sequence showing at least 50% similarity with the amino acid sequence as depicted in any of the sequences of complement inhibitors disclosed herein or a functional fragment thereof. The protein may comprise a polypeptide sequence which has at least 60%, or preferably at least 70%, more preferably 80%, more preferably, 90%, more preferably at least 99%, most preferably 100% similarity to the sequence of a complement inhibitor as described herein, or a functional fragment thereof.

The term “similarity” refers to a degree of similarity between proteins in view of differences in amino acids, but which different amino acids are functionally similar in view of almost equal size, lipophilicity, acidity, etc. is taken into account. A percentage similarity can be calculated by optimal alignment of the sequences using a similarity-scoring matrix such as the Blosum62 matrix described in Henikoff S. and Henikoff J. G., P.N.A.S. USA 1992, 89:10915-10919. Calculation of the percentage similarity and optimal alignment of two sequences using the Blosum62 similarity matrix and the algorithm of Needleman and Wunsch (J. Mol. Biol. 1970, 48:443-453) can be performed using the GAP program of the Genetics Computer Group (GCG, Madison, WI, USA) using the default parameters of the program.

Exemplary parameters for amino acid comparisons in the present invention use the Blosum62 matrix (Henikoff and Henikoff, supra) in association with the following settings for the GAP program:
Gap penalty: 8
Gap length penalty: 2
No penalty for end gaps.

Polymorphic forms of complement inhibitors described herein are included in the present disclosure. Variants of the complement inhibitors include natural or synthetic variants that may contain variations in the amino acid residue sequence due to deletions, substitutions, insertions, inversions or additions of one or more amino acid residues in said sequence or due to an alteration to a moiety chemically linked to a protein. For example, a protein variant may be an altered carbohydrate or PEG structure attached to a protein. The complement inhibitor may include at least one such protein modification.

Substitutional variants of proteins are those in which at least one amino acid residue in the amino acid sequence has been removed and a different amino acid residue inserted in its place. Protein complement inhibitors may contain conservative or non-conservative substitutions.

The term “conservative substitution”, relates to the substitution of one or more amino acid residues for amino acid residues having similar biochemical properties. Typically, conservative substitutions have little or no impact on the activity of a resulting protein. For example, a conservative substitution may be an amino acid residue substitution that does not substantially affect the ability of the protein to inhibit complement activity. Screening of variants of the protein complement inhibitors can be used to identify which amino acid residues can tolerate an amino acid residue substitution. In one example, the relevant biological activity of a modified protein is not decreased by more than 25%, preferably not more than 20%, especially not more than 10%, compared with the disclosed complement inhibitor when one or more conservative amino acid residue substitutions are effected.

One or more conservative substitutions can be included in a protein complement inhibitor. In one example, 10 or fewer conservative substitutions are included in the protein. A protein complement inhibitor may therefore include 1, 2, 3, 4, 5, 6, 7, 8, 9, 10 or more conservative substitutions. A polypeptide can be produced to contain one or more conservative substitutions by manipulating the nucleotide sequence that encodes that polypeptide using, for example, standard procedures such as site-directed mutagenesis, gene synthesis, or PCR. Alternatively, a polypeptide can be produced to contain one or more conservative substitutions by using peptide synthesis methods, for example as known in the art.

Examples of amino acid residues which may be substituted for an original amino acid residue in a protein and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val. In one embodiment, the substitutions are among Ala, Val Leu and Ile; among Ser and Thr; among Asp and Glu; among Asn and Gln; among Lys and Arg; and/or among Phe and Tyr. Further information about conservative substitutions can be found in, among other locations, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988), WO 00/67796 (Curd et al.) and in standard textbooks of genetics and molecular biology.

Other variants can be, for example, functional variants such salts, amides, esters, and specifically C-terminal esters, and N-acyl derivatives. Also included are peptides which are modified in vivo or in vitro, for example by glycosylation, amidation, carboxylation or phosphorylation.

Protein complement inhibitors can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR1R2 wherein R1 and R2 are each independently H or C1-C6 alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C1-C6 alkyl or dialkyl amino or further converted to an amide. Hydroxyl groups of the peptide side chains may be converted to alkoxy or ester groups, for example C1-C6 alkoxy or C1-C6 alkyl ester, using well-recognized techniques. Phenyl and phenolic rings of the peptide side chains may be substituted with one or more halogen atoms, such as F, Cl, Br or I, or with C1-C6 alkyl, C1-C6 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

The composition may comprise from 1 nM to 500 μM complement inhibitor. The composition may comprise from 1 nM to 100 μM complement inhibitor. The composition may comprise from 10 nM to 50 μM complement inhibitor. The composition may comprise from 1 nM to 5 μM complement inhibitor. The composition may comprise from 50 nM to 50 μM complement inhibitor. The composition may comprise from 50 nM to 1 μM complement inhibitor. The composition may comprise from 50 nM to 0.5 μM complement inhibitor. The composition may comprise from 50 nM to 0.1 μM complement inhibitor. The composition may comprise from 100 nM to 50 μM complement inhibitor. The composition may comprise from 250 nM to 50 μM complement inhibitor. The composition may comprise from 500 nM to 50 μM complement inhibitor. The composition may comprise from 750 nM to 50 UM complement inhibitor. The composition may comprise from 1 μM to 50 μM complement inhibitor.

The composition may comprise at least 1 nM complement inhibitor. The composition may comprise at least 10 nM complement inhibitor. The composition may comprise at least 50 nM complement inhibitor. The composition may comprise at least 100 nM complement inhibitor. The composition may comprise at least 250 nM complement inhibitor. The composition may comprise at least 500 nM complement inhibitor. The composition may comprise at least 750 nM complement inhibitor. The composition may comprise at least 1 μM complement inhibitor.

The composition may further comprise at least one biologically compatible salt. The biologically compatible salt may be a sodium salt, a calcium salt, or a magnesium salt. For example, the composition may comprise inorganic salts including sodium chloride (NaCl), calcium chloride (CaCl₂), or magnesium chloride (MgCl₂). The composition may comprise an organic salt including sodium lactate (NaCO₂CH(OH)CH₃) including sodium s-lactate and sodium l-lactate, sodium bicarbonate (NaHCO₃), sodium citrate (Na₃C₆H₅O₇) or sodium acetate (C₂H₃NaO₂).

The composition may further comprise a buffering agent. The buffering agent may be operable to maintain the pH of the composition or at least minimise changes in pH of the composition.

The composition may have an osmolarity of at least 200 milliosmoles per litre (mOsmol/L). The composition may have an osmolarity of at least 250 milliosmoles per litre (mOsmol/L). The composition may have an osmolarity of at least 250, 260, 280, 300, 320, 330, 340, 350, 360, 370, 380, 390, 400, 410, 420, 430, 450, 460, 470, 480, 490 or 500 mOsmol/L or values therebetween.

The composition may have an osmolarity of from 200 mOsmol/L to 500 mOsmol/L. The composition may have an osmolarity of from 250 mOsmol/L to 500 mOsmol/L. The composition may have an osmolarity of from 280 mOsmol/L to 500 mOsmol/L. The composition may have an osmolarity of from 300 mOsmol/L to 500 mOsmol/L. The composition may have an osmolarity of from 320 mOsmol/L to 500 mOsmol/L. The composition may have an osmolarity of from 250 mOsmol/L to 490 mOsmol/L. The composition may have an osmolarity of from 250 mOsmol/L to 480 mOsmol/L. The composition may have an osmolarity of from 250 mOsmol/L to 470 mOsmol/L. The composition may have an osmolarity of from 250 mOsmol/L to 460 mOsmol/L. The composition may have an osmolarity of from 250 mOsmol/L to 450 mOsmol/L. The composition may have an osmolarity of from 300 mOsmol/L to 480 mOsmol/L. The composition may have an osmolarity of from 300 mOsmol/L to 380 mOsmol/L. The composition may have an osmolarity of from 330 mOsmol/L to 370 mOsmol/L. The composition may have an osmolarity of from 450 mOsmol/L to 500 mOsmol/L. The composition may have an osmolarity of from 450 mOsmol/L to 490 mOsmol/L. The composition may have an osmolarity of from 460 mOsmol/L to 490 mOsmol/L. The composition may have an osmolarity of from 470 mOsmol/L to 490 mOsmol/L.

In a second aspect there is presented a composition for use in the inhibition of mesothelial cell transformation, the composition comprising a complement inhibitor.

Preferred and optional features of the complement inhibitor of the first aspect are preferred and optional features of the complement inhibitor of the second aspect.

According to a third aspect there is provided a method of making an enhanced composition for the use in peritoneal dialysis, the method comprising:
providing a base composition comprising an osmotic agent dissolved in an aqueous solvent;
providing a complement inhibitor;
adding the complement inhibitor to the base composition to form an enhanced composition.

Preferred and optional features of the complement inhibitor and osmotic agent of the first aspect are preferred and optional features of the complement inhibitor and osmotic agent of the third aspect.

In a fourth aspect there is provided a method of peritoneal treatment, the method comprising:
providing a composition according to the first aspect;
transferring the composition into a peritoneal cavity of a patient;
retaining the composition in the peritoneal cavity of the patient for a treatment time;
   removing the composition from the peritoneal cavity after the treatment time is completed;
   wherein the composition removed from the peritoneal cavity comprises toxins that have been drawn across the peritoneal membrane from the blood of the patient into the composition.

The treatment time may be an hour, two hours, three hours or more or values therebetween. The treatment time may be overnight.

According to a fifth aspect there is provided a composition for the use in peritoneal dialysis

(PD), the composition comprising an osmotic agent and a complement inhibitor.

The composition may be a dry composition and therefore may not include a solvent such as water, for example.

The composition may be a freeze-dried composition.

Prior to use, the user may add the composition to a solvent, such as water, for example, to form a peritoneal dialysis solution or fluid. The so-formed peritoneal dialysis solution or fluid may be the composition according to the first aspect. Accordingly, features of the composition of the first aspect are features of the composition of the fifth aspect when added to a solvent.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### BRIEF DESCRIPTION OF THE FIGURES

Embodiments of the present invention will now be described, by way of non-limiting example, with reference to the accompanying drawings.

FIG. 1. shows a schematic example of a patient undergoing peritoneal dialysis.

FIG. 2. shows the three pathways of the complement system, the various proteins involved and where therapeutic intervention can take place in the complement cascade, (Ricklin D et al. The renaissance of complement therapeutics. Nat Rev Nephrol 14(1), 26-47 (2018)).

FIG. 3. shows a schematic of plasmid pE-SUMO Kan, which was used to express SUMO-fusion proteins according to the present invention (http://www.lifesensors.com).

FIG. 4. Erythrocytes treated with AET to make ‘Paroxysmal nocturnal haemoglobinurea (PNH)-like’ cells that are susceptible to acidified serum lysis in a similar way to PNH erythrocytes. Adding increasing concentrations of PspCN inhibit lysis of the PNH-like erythrocytes with an IC50 of 27 nM. PspCN activates the FH already present in the serum and effectively prevents lysis.

FIG. 5. fluorescence-based confocal microscopy showing activation of complement by a PD solution on Human Mesothelial Cells (HMC).

FIG. 6. C3d levels measured as a marker of complement activation in pHUVECs exposed to a composite solution that includes 2.3% glucose PD solution (see Table 3 below), in the presence or not of increasing concentrations of the complement inhibitor FH. Results are shown for different cell passage numbers (P2-P6) and seeding times on the 24-well plates. A significant reduction in C3d levels is observed for all conditions except for P6, consistent with the primary cells likely having changed their genetic and phenotypic properties.

FIG. 7. C3d levels measured as a marker of complement activation in pHUVECs exposed to a composite solution that includes 2.3% glucose PD solution (see Table 3 below), in the presence or not of increasing concentrations of the complement inhibitor FH+PspCN complex. Results are shown for different cell passage numbers (P2-P6) and seeding times on the 24-well plates. A significant reduction in C3d levels is observed at P2, but not at P6 consistent with the primary cells likely having changed their genetic and phenotypic properties.

FIG. 8. C3d levels measured as a marker of complement activation in pHUVECs exposed to a composite solution that includes 2.3% glucose PD solution (see Table 3 below), in the presence or not of increasing concentrations of PspCN, an activity enhancer of the complement inhibitor FH. Results are shown for different cell passage numbers (P3-P5) and seeding times on the 24-well plates. A significant reduction in C3d levels is observed for all conditions. Of importance to note is the presence of 5% NHS in the composite solution which acts as source of complement but also contains FH that PspCN can bind to and activate thus increasing its potency.

FIG. 9. C3d levels measured as a marker of complement activation in pHUVECs exposed to a composite solution that includes 2.3% glucose PD solution (see Table 3 below), in the presence or not of increasing concentrations of the complement inhibitor DAF 1-4. Results are shown for different cell passage numbers (P3-P6) and seeding times on the 24-well plates. A significant reduction in C3d levels is observed for P3, less for P5 and no reduction for P6, consistent with the primary cells likely having changed their genetic and phenotypic properties.

FIG. 10. C5b-9 levels measured as a marker of complement activation in pHUVECs exposed to a composite solution that includes 2.3% glucose PD solution (see Table 3 below), in the presence or not of increasing concentrations of the complement inhibitors FH and FH+PspCN complex. Results are shown for cell passage numbers P2 and seeding time of 1 day on the 24-well plates. A marked reduction in C5b-9 levels is observed both for FH and FH+PspCN complex compared to no inhibitor-containing samples.

FIG. 11. C3d levels measured as a marker of complement activation in pHUVECs exposed to a composite solution that includes 2.3% glucose PD solution (see Table 3 below) at increasing concentrations. Approximately a 30% increase in C3d levels is observed in the solutions containing 95% PD solution (see Table 3 below) to 5% NHS vs. 95% cell media to 5% NHS.

FIG. 12. Microscope images of the pHUVECs exposed to composite solutions for 3 days at two magnifications. Cells exposed to 85% media to 10% PBS to 5% NHS maintain their characteristic cobblestone-like endothelial appearance whereas cells exposed to 35% media-40% PD solution (see Table 3 below) to 10% PBS to 5% NHS have started to lose their endothelial appearance and have become more elongated and spindle-like.

FIG. 13. Lactate dehydrogenase assay (LDH) data to measure toxicity of complement inhibitors on pHUVECs. The LDH assay is a means of measuring either the number of cells via total cytoplasmic LDH or membrane integrity as a function of the amount of cytoplasmic LDH released into the medium. The LDH shown correspond to LDH release normalized to total LDH/cell number. There is little difference observed between values obtained for cells exposed to their normal growth medium and values corresponding to cells exposed to their growth medium supplemented with the inhibitors.

FIG. 14. Gene Set Enrichment Analysis (GSEA) was used to identify genes involved in complement system and epithelial to mesenchymal transition (EMT) (A). 28 out of these 38 identified genes were found in transcriptome and proteome datasets from microdissected omental arterioles (Bartosova et al, JASN 2018) (B), including complement factors regulating the alternative pathway (highlighted in red).

FIG. 15. Exposure of primary human umbilican vein endothelial cells (pHUVEC) to TGFβ for 72 hours resulted in early EndMT. More spindle-like cells were observed in the culture (A) and expression of myofibroblast markers Col13A and alphaSMA was increased compared to medium controls (B). Mean±SD are shown, scale bar=200 μm, *p<0.05, ****p<0.0001.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Example compositions suitable for use in peritoneal dialysis, compositions according to the disclosure, uses of the compositions according to the disclosure and methods of treatment using the compositions are described below.

### Comparative Example 1

A first example typical peritoneal dialysis composition has a composition according to Table 1.

TABLE 1

| Component | Concentration (g/L) |
|---|---|
| Icodextrin | 75 |
| Sodium chloride | 5.4 |
| Sodium S-Lactate | 4.5 |
| Calcium Chloride | 0.26 |
| Magnesium chloride | 0.05 |

The composition has a theoretical osmolarity of 284 milliosmoles per litre (mOsmol/L) and pH from 5 to 6.

The composition is used in a peritoneal dialysis treatment as either part of a continuous ambulatory peritoneal dialysis (CAPD) or automated peritoneal dialysis (APD) for the treatment of chronic renal failure.

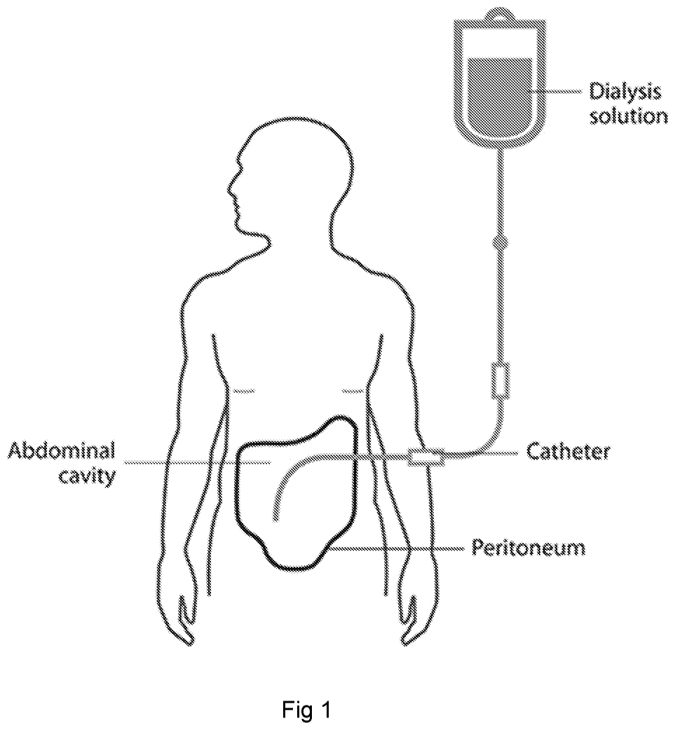

A schematic view of a typical peritoneal dialysis treatment is shown in FIG. 1. Typically, during a treatment the composition is transferred into the peritoneal cavity of a patient via a catheter installed into the patient's abdomen. The composition is retained within the peritoneal cavity for either overnight (6-12 hours) in CAPD treatments, or 14 to 16 hours in APD treatments.

Typical volumes of peritoneal dialysis compositions used in a treatment is up to 2 L of fluid administered over a period of 10 to 20 minutes for average patient size.

After a normal treatment time the fluid is removed from the peritoneal cavity.

### Comparative Example 2

A second example typical peritoneal dialysis composition has a composition according to Table 2.

TABLE 2

| Component | Concentration (g/L) |
|---|---|
| Glucose monohydrate | 25.00 |
| Equivalent to Anhydrous glucose | 22.70 |
| Sodium chloride | 5.38 |
| Sodium S-Lactate | 1.68 |
| Calcium Chloride | 0.18 |
| Magnesium chloride | 0.05 |
| Sodium bicarbonate | 2.10 |

The composition has an osmolarity of 395 milliosmoles per litre (mOsmol/L) and pH of 7.4.

The composition is used in a peritoneal dialysis treatment as either part of a continuous ambulatory peritoneal dialysis (CAPD) or automated peritoneal dialysis (APD) for the treatment of chronic renal failure.

Typically, during a treatment the composition is transferred into the peritoneal cavity of a patient via a catheter installed into the patient's abdomen. The composition is retained within the peritoneal cavity for 4 cycles per day in CAPD treatments, or during 4-5 cycles at night and up to 2 cycles during the day in APD treatments.

Typical volumes of peritoneal dialysis compositions used in a treatment is up to 2 L of fluid administered over a period of 10 to 20 minutes for average patient size.

After a normal treatment time the fluid is removed from the peritoneal cavity.

### The Role of the Alternative Complement Pathway in the Transformation of Mesothelial Cells

It has been suggested that the transformation of mesothelial cells is involved in the degradation of the peritoneal membrane. Accordingly, the expression of C3, CFB, C5b9 and C1q was investigated in human mesothelial cells (HMC).

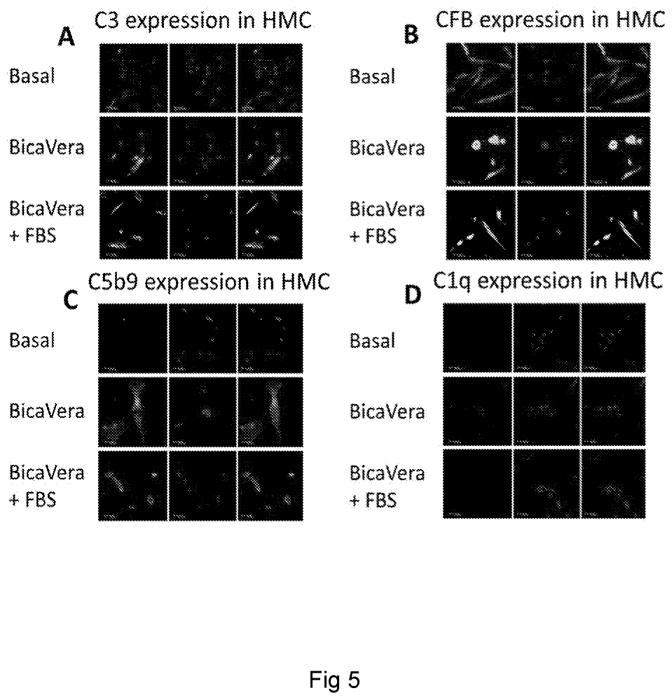

Immortalized HMC were grown as monolayers and incubated for 8 h with BicaVera PD solution (Fresenius) which contains 2.5% glucose. After incubation, the cells were exposed to Fetal Bovine Serum (FBS) for 1 h and then incubated with the antibodies. Specific fluorescence was evaluated by confocal microscopy showing C3 (FIG. 5A), CFB (FIG. 5B), C5b9 (FIG. 5C) and C1q (FIG. 5D) expression by HMC. For each of FIGS. 5A-D the top 3 images (basal) show blue staining which corresponds to cell death markers, the middle 3 images show the colocalization of blue and green staining (merged), where green staining shows expression of the corresponding complement activation markers after incubation with the BicaVera solution, and the bottom 3 images show the colocalization of cell death and complement activation markers after incubation with the BicaVera solution in the presence of FBS.

Acquisition of staining for C3, CFB and C5b9 after incubation of the HMC with the BicaVera PD solution demonstrates that simple contact with glucose can induce local expression and activation of the alternative pathway of complement. This occurs even in the absence of FBS indicating that complement markers are made locally by the cells and not the FBS. There is not much evidence of classical pathway activation as seen in FIG. 5D. These results demonstrate the importance of local complement pathway activation in mesothelial cell transformation and highlight the need for complement regulation.

### Example 1

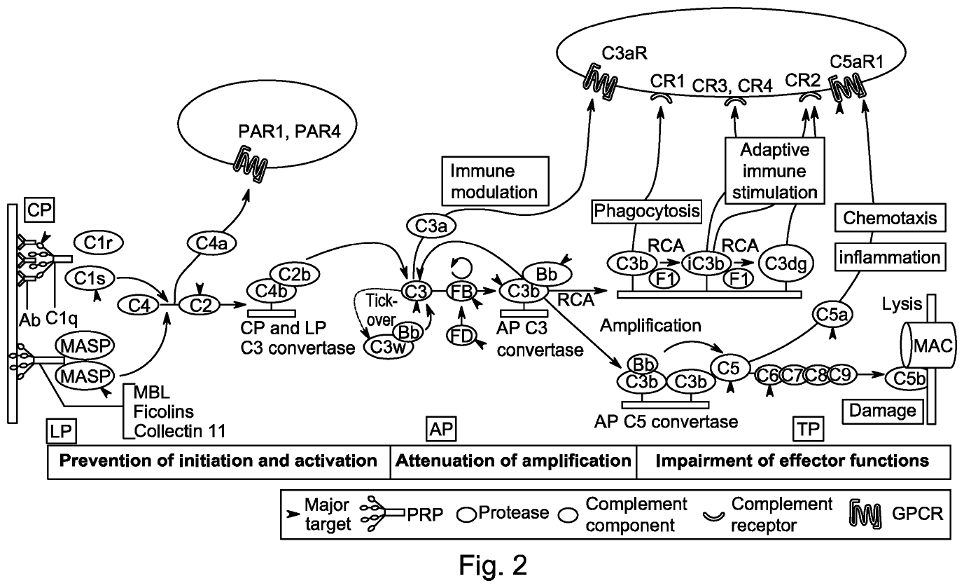

With reference to FIG. 2, the complement system can be activated by three pathways, the classical pathway (CP), the mannose-binding lectin pathway (LP) and the alterative pathway (AP). All three pathways converge at the cleavage of C3 to C3b, leading to the amplification of the initial response through the AP and interaction of C3b with factor B (FB) and factor D (FD) to form new C3 convertases. When regulators of complement activation (RCAs) are absent the C3 convertases react with further C3b molecules to form the C5 convertases. These cleave C5 and initiate the events that lead to the formation of the membrane attack complex (MAC). The C3 and C5 convertases lead to the release of the anaphylatoxins C3a and C5a which can initiate downstream inflammatory responses. RCAs attenuate the assembly of the convertases and act as cofactors to factor I to degrade C3b to iC3b and C3d which remain attached to the surface and can stimulate phagocytosis and/or immune signalling. Complement therapeutic agents can act by preventing initiation, by attenuating amplification or affecting downstream effector functions. Complement inhibition can be targeted towards FB, FD, Properdin, MASPs 1 to 3, C1, C3, C3a, C3b, C5, C5a, C5b, C5aR1, C6 or MAC. Alternatively, enhancers or activators of the RCAs can be used.

An example composition according to the disclosure is the composition of comparative example 1 including 0.5 μM of the complement inhibitor PspCN (SEQ ID NO 1), characterised below.

It is known that PspCN is an especially effective inhibitor of C3b and C3d as demonstrated below. Accordingly, a composition comprising PspCN in a peritoneal dialysis (PD) treatment will inhibit the activation of the complement immune response in the peritoneal cavity and peritoneal membrane, thereby inhibiting the structural changes of the peritoneal membrane that have been reported over prolonged PD treatment.

A further example composition is the composition of comparative example 2 including 1.5 μM of the complement inhibitor DAF 1-4 (SEQ ID NO: 12).

### Example Complement Inhibitor PspCN

The complement inhibitor PspCN has been described and the ability of PspCN to inhibit the alternative complement pathway has been demonstrated in International patent application WO 2015/055991 to the University Court to the University of Edinburgh, the contents of which is herein incorporated by reference. A summary of the findings presented therein is provided below:

### Preparation of Protein

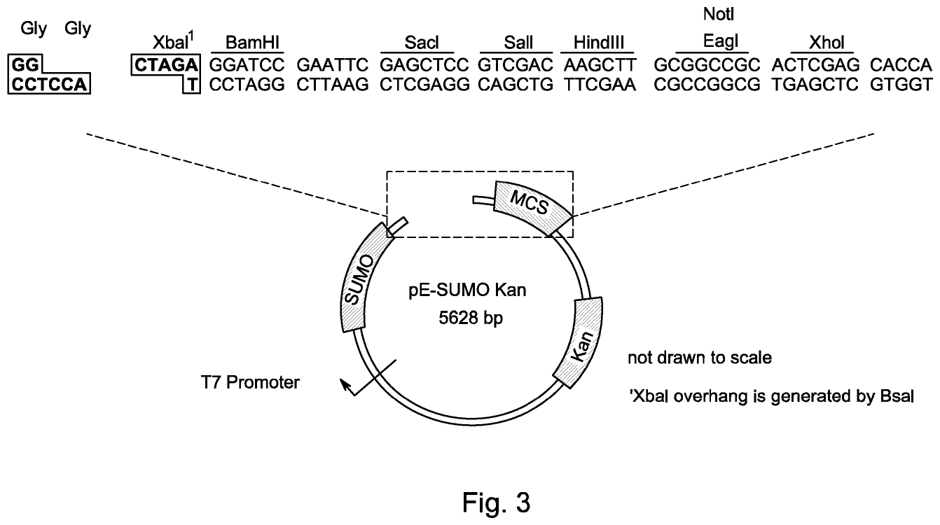

A gene representing residues 37-140 of PspC (D39) was optimised for expression in Escherichia coli (see SEQ ID NO 4 below) and purchased from GeneArt-LifeTech. The resulting construct was then cloned into the pE-SumoProKan E. coli expression vector (LifeSensors, Malvern, PA, see FIG. 3) and expressed in BL21 (DE3) E. coli in lysogeny broth (LB). Protein production was induced overnight at 25° C. by the addition of 0.25 mM isopropyl β-D-1-thiogalactopyranoside (IPTG). The resultant hexaHis-SUMO-tagged protein was named “sPspCN” and was captured on a His Trap immobilised Ni²⁺-affinity column (GE Healthcare) and eluted with a linear gradient of 0-0.5 M imidazole. Samples of sPspCN were further purified by size-exclusion chromatography on a HiPrep Superdex 75 column (GE Healthcare) equilibrated with phosphate-buffered saline (PBS). A similar strategy was used to prepare a longer construct, embracing the adjacent R1 domain (see FIG. 2), (residues 37-292) called sPspCNR1.

For most experiments, the catalytic domain from the SUMO-specific protease, ULP1, was used to remove the hexaHis-SUMO tags from sPspCN (or sPspCNR1) (with no vector-derived residues), following which the tag was removed by a second Ni²⁺-affinity chromatographic step. The cleaved material—PspCN (or PspCNR1)—was then purified further on a HiPrep Superdex 75 column in PBS as above. Proteins were judged to be homogeneous by SDS-PAGE and the integrity and identity of the proteins was confirmed by mass spectrometry (not shown).

### Demonstration That PspCN can Activate Factor H Present in a Serum and Effectively Prevent Cell Lysis

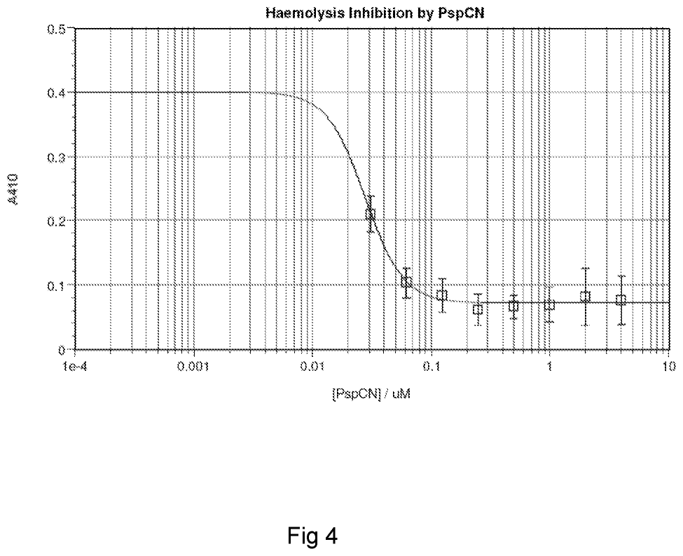

With reference to FIG. 4, it has been shown that addition of PspCN (SEQ ID NO. 1) to PNH-like cells inhibits lysis of the cells with IC50 of 27 nM. The PspCN activates the factor H already present in the serum with the cells and thereby prevents complement induce lysis of the cells.

Accordingly, PspCN is a suitable complement inhibitor for use in a PD composition as described above.

### Example Complement Inhibitor DAF 1-4

### Preparation of Protein

A gene comprising complement control protein modules 1-4 from human Decay Acceleration Factor (DAF 1-4) was optimised for expression in Komagataella phaffii (colloquially Pichia Pastoris) and inserted into the pPICZalphaB expression vector by Thermofisher Scientific. Proprietary KM71 Komagataella phaffii, designated KM71 PDI, was transformed to express DAF 1-4 by electroporation and successfully transformed colonies were selected from Zeocin® agar plates.

Selected colonies were grown to increased cell number in Buffered Complex Glycerol Medium (BMGY) in baffled shaker flasks (30° C., 225 RPM) over a period of 64-72 hours.

Cells were reconstituted into Buffered Complex Methanol Medium (BMMY) to effect protein production/excretion. Growth in BMMY proceeded over 96 hours at in baffled shaker flasks (20° C., 225 RPM) with 0.5% (v/v) supplementary neat methanol being applied twice daily until harvested.

The produced clarified supernatant was formulated to 1 mM phenylmethylsulfonyl fluoride (pmsf) with 5 mM ethylenediaminetetraacetic acid (EDTA) then via continuous volume diafiltration was buffer exchanged into Dulbecco's phosphate buffered saline (DPBS). Protein purification was conducted using Capto™ SP ImpRes cation exchange media by utilising a citric acid buffer system at pH 4.5 with a linear salt gradient from 0-500 mM NaCl. Final polishing was conducted by size exclusion chromatography (SEC) using Superdex 75 media and isocratic elution into DPBS.

Analysis of intermediates was conducted via SDS-PAGE and analytical SEC (TOSOH g3000SWXL column) with confirmation of the integrity and identity of the protein being confirmed by the same, with inclusion of mass spectrometry (not shown).

### Method Description for Cell Culture and Exposure to PD Solution and Complement Inhibitors

Primary Human Umbilical Vein Endothelial Cells (pHUVEC, PromoCell) were grown in media supplemented with 1% penicillin/streptomycin according to the manufacturer's instructions in a 5% CO₂ incubator. Cells from passages 2-6 (P2-P6) were used for the C3d assays and were seeded either overnight or for 5-7 days in 24-well ELISA plates. The passage number is important as after P5 the cells lose their primary characteristics (genetic and phenotypic properties). Cells were subsequently exposed to a commercially available 2.3% glucose PD solution (see Table 3 below)—cell media composite+5% *NHS (Normal human serum, used as a source of complement)+6-20% PBS (to keep the PBS content from the inhibitor stock solution as a constant), ±complement inhibitors (e.g. DAF 1-4, FH, PspCN) for 20-24 hours.

TABLE 3
Commercial PD Solution

| Component | Concentration (g/L) |
|---|---|
| Glucose monohydrate | 25.00 |
| Equivalent to Anhydrous glucose | 22.70 |
| Sodium chloride | 5.64 |
| Sodium S-Lactate | 3.93 |
| Calcium Chloride | 0.18 |
| Magnesium chloride | 0.10 |

Cells were fixed in methanol at −20° C. for 7-10 min, washed with PBS, permeabilized with 0.2% TritonX-100 for 8-10 min, blocked with Superblock-PBS and incubated with primary C3d antibody (Abcam). Alexa 594 (Abcam) secondary antibody+DAPI stain (SIGMA; nuclei staining, indicator of cell number) were added and fluorescence measurements were performed using a CLARIOstar Plus plate reader. Data were normalized to DAPI stain and subsequently to the maximum value-assay background within each experiment. C3d is the final surface-adhering part of C3b following complement activation and was used as the biomarker for measuring complement activation.

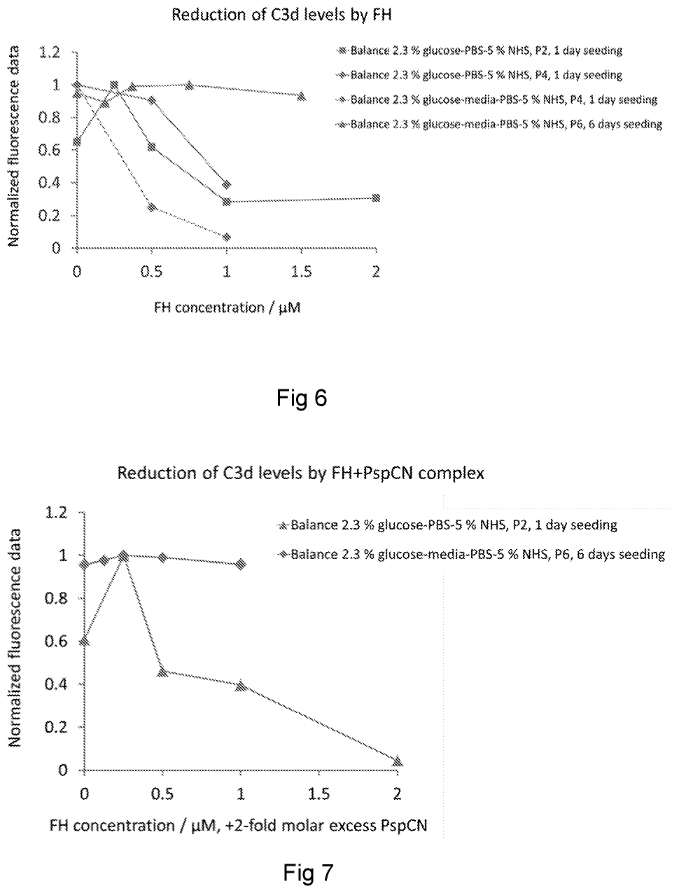

The results are shown in FIGS. 6-9.

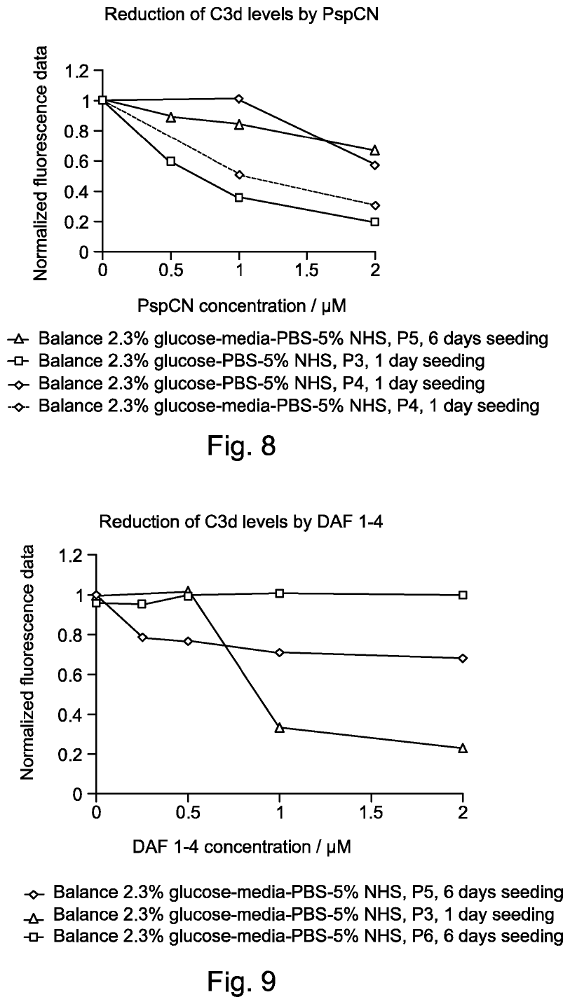

A significant reduction in C3d levels is observed in FIG. 6 for all conditions except for P6, which is consistent with the primary cells likely having changed their genetic and phenotypic properties. A significant reduction in C3d levels is observed in FIG. 7 at P2, but not at P6 consistent with the primary cells likely having changed their genetic and phenotypic properties. A significant reduction in C3d levels is observed for all conditions in FIG. 8. Of importance to note is the presence of 5% NHS in the composite solution which acts as source of complement but also contains FH that PspCN can bind to and activate thus increasing its potency. A significant reduction in C3d levels is observed in FIG. 9 for P3, less for P5 and no reduction for P6, consistent with the primary cells likely having changed their genetic and phenotypic properties.

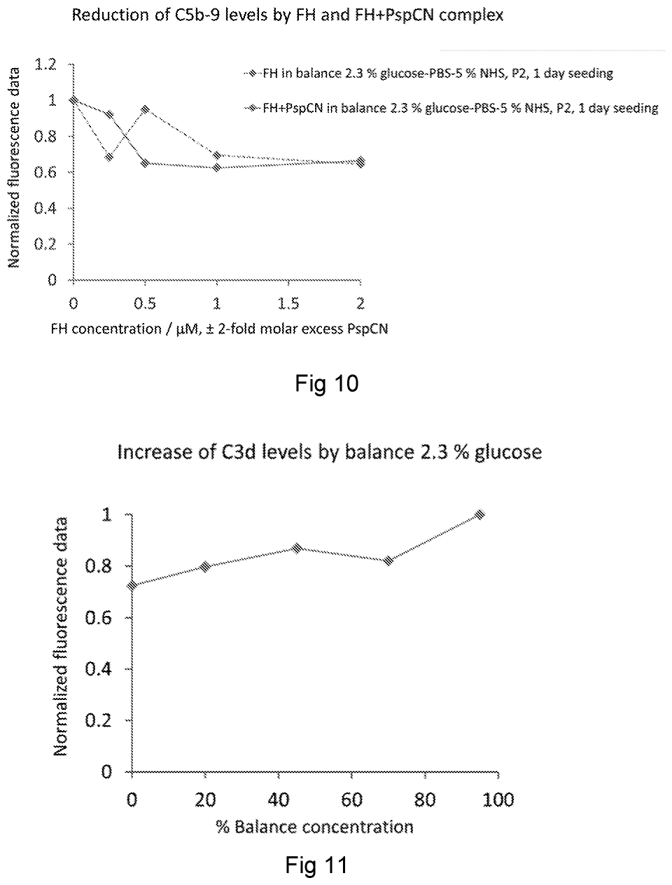

C5b-9 levels were measured in a similar assay as described above for C3d. Results are shown in FIG. 10 for cell passage numbers P2 and seeding time of 1 day on the 24-well plates. A marked reduction in C5b-9 levels is observed both for FH and FH+PspCN complex compared to no inhibitor-containing samples.

FIG. 11 shows that C3d levels increase by approximately 30% in the solutions containing 95% balance 2.3% glucose-5% NHS vs. 95% cell media-5% NHS.

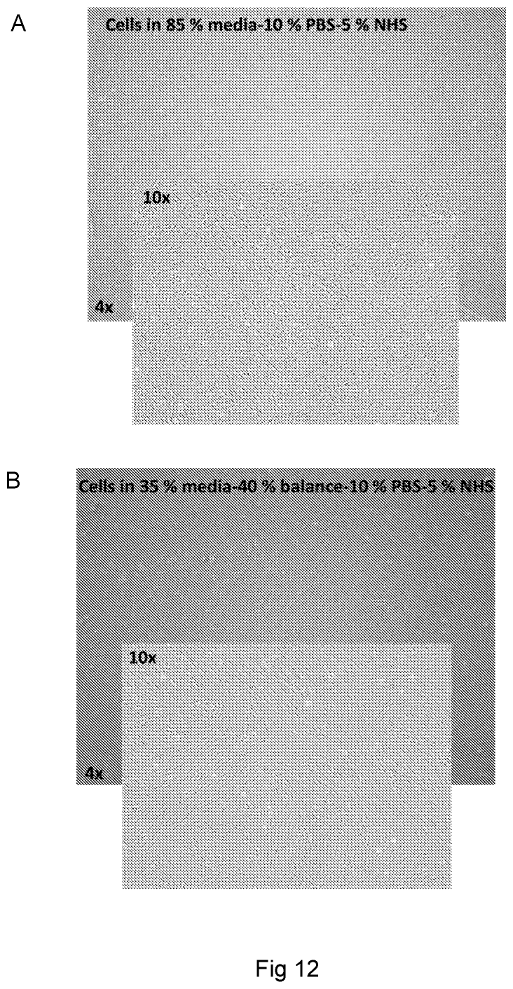

Physiological changes in pHUVEC cells have been shown to be associated with exposure to a PD solution. FIG. 12 shows microscope images of pHUVECs exposed to composite solutions for 3 days at two magnifications (4 times and 10 times). Cells exposed to 85% media-10% PBS-5% NHS are shown to maintain their characteristic cobblestone-like endothelial appearance (FIG. 12A) whereas cells exposed to 35% media-40% balance 2.3% glucose-10% PBS-5% NHS have started to lose their endothelial appearance and have become more elongated and spindle-like (FIG. 12B).

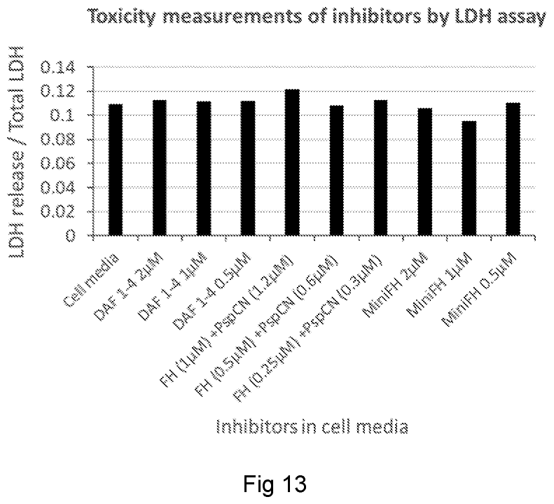

The toxicity of complement inhibitors on pHUVEC cells was measured using a Lactate dehydrogenase (LDH) assay. A LDH assay is a means of measuring either the number of cells via total cytoplasmic LDH or membrane integrity as a function of the amount of cytoplasmic LDH released into the medium. The LDH shown correspond to LDH release normalized to total LDH/cell number. The results are shown in FIG. 13. Little difference is observed between values obtained for cells exposed to their normal growth medium and values corresponding to cells exposed to their growth medium supplemented with the inhibitors. Accordingly, the complement inhibitors tested had no discernible toxicity on the pHUVEC cells.

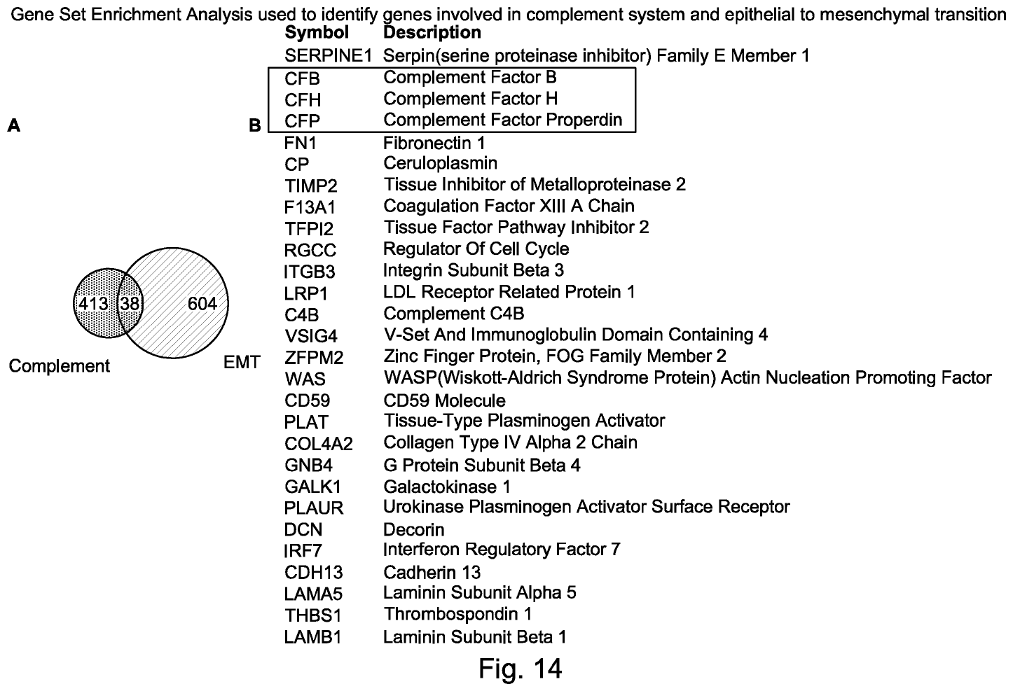

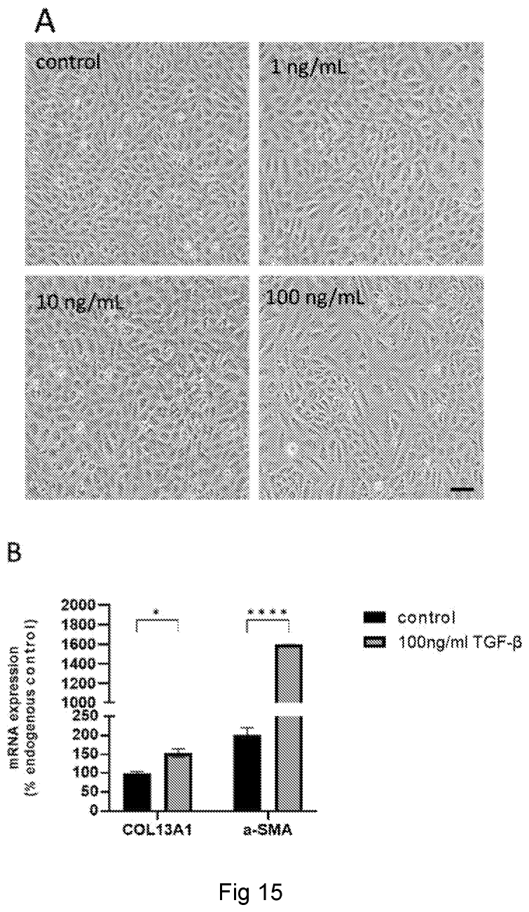

It has been found that upon exposure to low glucose degradation products (GDP) PD solution 604 genes are upregulated in cells, 38 of these genes are involved in the complement system thereby providing a link between complement and the epithelial to mesenchymal transition (EMT) (see FIG. 14). Factor H (FH), factor B (FB) and properdin (factor P) were amongst the genes identified, highlighting the involvement of the alternative pathway of complement. Accordingly, the use of complement inhibitors for use in PD is expected to mitigate the epithelial to mesenchymal transition for cells exposed to the PD solution during PD treatment. Exposure of pHUVECs to TGFB for 72 hours resulted in early EMT (TGFβ is used to stimulate EMT, see FIG. 15A). Expression of myofibroblast markers Col13A and alphaSMA was increased compared to medium controls (see FIG. 15B). This increase relates to mesenchymal phenotype acquisition. It is anticipated that inclusion of the complement inhibitors in that setting would reduce early EMT, which would result in a reduction of the levels corresponding to those markers when compared to a system lacking complement inhibitors.

Accordingly, it has been shown that exposure to peritoneal dialysis solution can induce physiological changes in cells that are similar to those that have been associated with the EMT. Further, it has been shown that complement inhibitors such as FH, PspCN and DAF 1-4 can inhibit this transition. Therefore, it can be expected that in a clinical application, a peritoneal dialysis solution that comprises a complement inhibitor will at least reduce the EMT in the endothelial cells of the peritoneum to thereby extend the period for which peritoneal dialysis may be effectively used.

Amino acid sequence of PspC of strain D39 (NCTC no 7466) of S. pneumoniae is shown below (SEQ ID NO 7) and the PspCN sequence (amino acids 37 to 140) is shown in bold:

MFASKSERKVHYSIRKFSIGVASVAVASLVMGSVVHATENEGSTQAATS
SNMAKTEHRKAAKQVVDEYIEKMLREIQLDRRKHTQNVALNIKLSAIKT
KYLRELNVLEEKSKDELPSEIKAKLDAAFEKFKKDTLKPGEKVAEAKKK
VEEAKKKAEDQKEEDRRNYPTNTYKTLELEIAEFDVKVKEAELELVKEE
AKESRNEGTIKQAKEKVESKKAEATRLENIKTDRKKAEEEAKRKADGKL
KEANVATSDOGKPKGRAKRGVPGELATPDKKENDAKSSDSSVGEETLPS
SSLKSGKKVAEAEKKVEEAEKKAKDQKEEDRRNYPTNTYKTLDLEIAES
DVKVKEAELELVKEEAKEPRDEEKIKQAKAKVESKKAEATRLENIKTDR
KKAEEEAKRKAAEEDKVKEKPAEQPQPAPATQPEKPAPKPEKPAEQPKA
EKTDDQQAEEDYARRSEEEYNRLTQQQPPKTEKPAQPSTPKTGWKQENG
MWYFYNTDGSMATGWLQNNGSWYYLNANGAMATGWLQNNGSWYYLNANG
SMATGWLQNNGSWYYLNANGAMATGWLQYNGSWYYLNSNGAMATGWLQY
NGSWYYLNANGDMATGWLQNNGSWYYLNANGDMATGWLQYNGSWYYLNA
NGDMATGWVKDGXTWYYLKASGAMKASQWFKVSDKWYYVNGSGALAVNT
TVDGYGVNANGEWVN

The synthetic, codon-optimised DNA sequence that was used for the expression of PspCN is shown below (SEQ ID NO 8):

GCAACCGAAAATGAAGGTAGCACCCAGGCAGCAACCAGCAGCAATATGG
CAAAAACCGAACATCGTAAAGCAGCCAAACAGGTTGTGGATGAGTATAT
CGAAAAAATGCTGCGTGAAATTCAGCTGGATCGTCGTAAACATACCCAG
AATGTTGCACTGAACATTAAACTGAGCGCCATCAAAACCAAATATCTGC
GTGAACTGAATGTGCTGGAAGAGAAAAGCAAAGATGAACTGCCGAGCGA
AATTAAAGCAAAACTGGATGCAGCCTTTGAAAAATTCAAAAAAGATACC
CTGAAACCGGGTGAGAAATAA

While there has been hereinbefore described approved embodiments of the present invention, it will be readily apparent that many and various changes and modifications in form, design, structure and arrangement of parts may be made for other embodiments without departing from the invention and it will be understood that all such changes and modifications are contemplated as embodiments as a part of the present invention as defined in the appended claims.

1. A composition for the use in peritoneal dialysis (PD), the composition comprising a biologically compatible solvent, an osmotic agent and a complement inhibitor.

2. The composition of claim 1, wherein the biologically compatible solvent is water and the composition is an aqueous composition.

3. The composition of claim 1, wherein the osmotic agent is selected from the group consisting of: glucose, dextrose (L-glucose), fructose, galactose, maltose, xylitol, mannitol, sorbitol, maltodextrin, icodextrin, sucrose, hyaluronic acid, or derivations or fragments or mixtures thereof.

4. The composition of claim 3, wherein the osmotic agent is selected from the group consisting of: glucose, dextrose, or icodextrin or derivations or fragments or mixtures thereof.

5. The composition of claim 1, wherein the complement inhibitor is an inhibitor of Factor D, Factor B, Properdin, MASPs1-3, C1, C3, C3a, C3b, C4b, C5, C5a, C5b, C5aR1, C6 or MAC.

6. The composition of claim 5, wherein the complement inhibitor accelerates the decay of the C3 convertase.

7. The composition of claim 5, wherein the complement inhibitor is selected from the group consisting of: C1-INH; IFX-1/CaCP29; Mirococept or APT070; TP10/CDX-1135; Eculizumab; AMY-101; Ravulizumab or ALX1210 or Ultomiris; Crovalimab/SKY59/RO7112689; Tesidolumab/LFG316;Pozelimab/REGN3918; ABP959; SB12; Nomacopan/rVA576/Coversin/OmCI; Zilucoplan/RA101495; Cemdisiran/ALN-CC5; APL-2; LNP023; Danicopan/ACH-4471/ACH-0144471; Sutimlimab/BIV009/TNT009; Avacopan/CCX168; Narsoplimab/OMS721; Zimura/avacincaptad pegol; Lampalizumab; CLG561; IONIS-FB-LRx; IPH5401; GEN1029; Ruconest; ACH-5228; ACH-5448; APL-9; AAVCAGsCD59/HMR59; ANX005; ANX007; BIVV020; OMS906; PRO-02; AMY-103; 5C6/Compsorbin; anti-FH.07; AMY-201/miniFH; variant mini FH (SEQ ID NO:9); DAF 1-4; SOBI005; ISU305; Mubodima; IFX-2; IFX-3; ALS-205; DF2593A; Regenemab; C6-LNA; or PspC or functional variant or fragment thereof.

8. The composition of claim 7, wherein the complement inhibitor prevents the initiation and amplification of complement and is selected from a group consisting of: C1-INH; Sutimlimab/BIV009/TNT009; Narsoplimab/OMS721; Ruconest; ANX005; ANX007; BIVV020; PRO-02.

9. The composition of claim 7, wherein the complement inhibitor attenuates the amplification of complement and is selected from a group consisting of: Mirococept (APT070); TP10/CDX-1135 (soluble complement receptor 1); AMY-101; APL-2; LNP023; Danicopan/ACH-4471/ACH-0144471; Sutimlimab/BIV009/TNT009; Lampalizumab; CLG561; IONIS-FB-LRx; ACH-5228; ACH-5448; APL-9; BIVV020; OMS906; PRO-02; AMY-103; 5C6/Compsorbin; anti-FH.07; AMY-201/miniFH; DAF 1-4; variant mini FH (SEQ ID NO:9); or PspC or functional variant or fragment thereof.

10. The composition of claim 9, wherein the complement inhibitor is a protein capable of binding to complement factor H.

11. The composition of claim 7, wherein the complement inhibitor impairs effector functions of complement and is selected from a group consisting of: IFX-1/CaCP29; Eculizumab (Soliris); Ravulizumab/ALX1210/Ultomiris; Crovalimab/SKY59/RO7112689; Tesidolumab/LFG316; Pozelimab/REGN3918; ABP959; SB12; Nomacopan/rVA576/Coversin/OmCI; Zilucoplan/RA101495; Cemdisiran/ALN-CC5; Zimura/avacincaptad pegol; Lampalizumab; CLG561; IONIS-FB-LRx; IPH5401; GEN1029; AAVCAGsCD59/HMR59; SOBI005; ISU305; Mubodima; IFX-2; IFX-3; ALS-205; DF2593A; Regenemab; C6-LNA.

12. The composition of claim 1, wherein the complement inhibitor is vaccina virus complement control protein (VCP), or smallpox inhibitor of complement enzymes (SPICE) or monkeypox virus inhibitor of complement enzymes (MOPICE), or a functional fragment or variant thereof.

13. The composition of claim 1, further comprising at least one biologically compatible salt.

14. The composition of claim 13, wherein the biologically compatible salt is a sodium salt, calcium salt, or a magnesium salt.

15. The composition of claim 1, wherein the composition has an osmolarity of at least 250 milliosmoles per litre (mOsmol/L).

16. The composition of claim 15, wherein the composition has an osmolarity of at least any of the following: 260, 280, 300, 320, 330, 340, 350, 360, 370, 380, 390, 400, 410, 420, 430, 450, or 500 mOsmol/L.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method of making an enhanced composition for the use in peritoneal dialysis, the method comprising:
providing a base composition comprising an osmotic agent dissolved in an aqueous solvent;
providing a complement inhibitor;
adding the complement inhibitor to the base composition to form an enhanced composition.

32. The method of claim 31, wherein the complement inhibitor is an inhibitor of Factor D, Factor B, Properdin, MASPs1-3, C1, C3, C3a, C3b, C4b, C5, C5a, C5b, C5aR1, C6 or MAC.

33. The composition of claim 32, wherein the complement inhibitor accelerates the decay of the C3 convertase.

34. The method of claim 31, wherein the complement inhibitor is selected from the group consisting of: C1-INH; IFX-1/CaCP29; Mirococept or APT070; TP10/CDX-1135; Eculizumab; AMY-101; Ravulizumab or ALX1210 or Ultomiris; Crovalimab/SKY59/RO7112689; Tesidolumab/LFG316; Pozelimab/REGN3918; ABP959; SB12; Nomacopan/rVA576/Coversin/OmCl; Zilucoplan/RA101495; Cemdisiran/ALN-CC5; APL-2; LNP023; Danicopan/ACH-4471/ACH-0144471; Sutimlimab/BIV009/TNT009; Avacopan/CCX168; Narsoplimab/OMS721; Zimura/avacincaptad pegol; Lampalizumab; CLG561; IONIS-FB-LRx; IPH5401; GEN1029; Ruconest; ACH-5228; ACH-5448; APL-9; AAVCAGsCD59/HMR59; ANX005; ANX007; BIVV020; OMS906; PRO-02; AMY-103; 5C6/Compsorbin; AMY-201/miniFH; variant mini FH (SEQ ID NO:9); DAF 1-4; SOBI005; ISU305; Mubodima; IFX-2; IFX-3; ALS-205; DF2593A; Regenemab; C6-LNA; or PspC or functional variant or fragment thereof.

35. A method of peritoneal treatment, the method comprising:
providing a composition according to claim 1;
transferring the composition into a peritoneal cavity of a patient;
retaining the composition in the peritoneal cavity of the patient for a treatment time;
removing the composition from the peritoneal cavity after the treatment time is completed;
wherein the composition removed from the peritoneal cavity comprises toxins that have been drawn across the peritoneal membrane from the blood of the patient into the composition.


SEQUENCE LISTING (12 sequences)

SEQ ID NO: 1 - protein, 104 aa, Streptococcus pneumoniae
ATENEGSTQAATSSNMAKTEHRKAAKQVVDEYIEKMLREIQLDRRKHTQNVALNIKLSAIKTKYLRELNVLEEKSKDELPSEIKAKLDAAFEKFKKDTLKPGEK

SEQ ID NO: 2 - protein, 79 aa, Streptococcus pneumoniae
KQVVDEYIEKMLREIQLDRRKHTQNVALNIKLSAIKTKYLRELNVLEEKSKDELPSEIKAKLDAAFEKFKKDTLKPGEK

SEQ ID NO: 3 - protein, 143 aa, Streptococcus pneumoniae
ATENEGATQVPTSSNRANESQAEQGEQPKKLDSERDKARKEVEEYVKKIVGESYAKSTKKRHTITVALVNELNNIKNEYLNKIVESTSESQLQILMMESRSKVDEAVSKFEKDSSSSSSSDSSTKPEASDTAKPNKPTEPGEK

SEQ ID NO: 4 - protein, 244 aa, Vaccinia virus
CCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 5 - protein, 244 aa, Variola virus
CCTIPSRPINMKFKNSVETDANANYNIGDTIEYLCLPGYRKQKMGPIYAKCTGTGWTLFNQCIKRRCPSPRDIDNGHLDIGGVDFGSSITYSCNSGYYLIGEYKSYCKLGSTGSMVWNPKAPICESVKCQLPPSISNGRHNGYNDFYTDGSVVTYSCNSGYSLIGNSGVLCSGGEWSNPPTCQIVKCPHPTILNGYLSSGFKRSYSYNDNVDFTCKYGYKLSGSSSSTCSPGNTWQPELPKCVR

SEQ ID NO: 6 - protein, 200 aa, Monkeypox virus
YCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGKLLAA

SEQ ID NO: 7 - protein, 701 aa, Streptococcus pneumoniae
MFASKSERKVHYSIRKFSIGVASVAVASLVMGSVVHATENEGSTQAATSSNMAKTEHRKAAKQVVDEYIEKMLREIQLDRRKHTQNVALNIKLSAIKTKYLRELNVLEEKSKDELPSEIKAKLDAAFEKFKKDTLKPGEKVAEAKKKVEEAKKKAEDQKEEDRRNYPTNTYKTLELEIAEFDVKVKEAELELVKEEAKESRNEGTIKQAKEKVESKKAEATRLENIKTDRKKAEEEAKRKADGKLKEANVATSDQGKPKGRAKRGVPGELATPDKKENDAKSSDSSVGEETLPSSSLKSGKKVAEAEKKVEEAEKKAKDQKEEDRRNYPTNTYKTLDLEIAESDVKVKEAELELVKEEAKEPRDEEKIKQAKAKVESKKAEATRLENIKTDRKKAEEEAKRKAAEEDKVKEKPAEQPQPAPATQPEKPAPKPEKPAEQPKAEKTDDQQAEEDYARRSEEEYNRLTQQQPPKTEKPAQPSTPKTGWKQENGMWYFYNTDGSMATGWLQNNGSWYYLNANGAMATGWLQNNGSWYYLNANGSMATGWLQNNGSWYYLNANGAMATGWLQYNGSWYYLNSNGAMATGWLQYNGSWYYLNANGDMATGWLQNNGSWYYLNANGDMATGWLQYNGSWYYLNANGDMATGWVKDGXTWYYLKASGAMKASQWFKVSDKWYYVNGSGALAVNTTVDGYGVNANGEWVN

SEQ ID NO: 8 - DNA, 315 bp, synthetic construct
  source 1..315: mol_type = other DNA
  misc_feature 1..315: note = Synthetic codon-optimised DNA sequence that was used for the expression of PspCN
gcaaccgaaaatgaaggtagcacccaggcagcaaccagcagcaatatggcaaaaaccgaacatcgtaaagcagccaaacaggttgtggatgagtatatcgaaaaaatgctgcgtgaaattcagctggatcgtcgtaaacatacccagaatgttgcactgaacattaaactgagcgccatcaaaaccaaatatctgcgtgaactgaatgtgctggaagagaaaagcaaagatgaactgccgagcgaaattaaagcaaaactggatgcagcctttgaaaaattcaaaaaagataccctgaaaccgggtgagaaataa

SEQ ID NO: 9 - protein, 502 aa, synthetic construct
  REGION 1..502: note = Edinburgh mini FH
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLGGGSGGGGSGGGTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 10 - DNA, 56 bp, synthetic construct
  source 1..56: mol_type = other DNA
  misc_feature 1..56: note = Portion of pE-SUMO Kan plasmid sequence
ctagaggatccgaattcgagctccgtcgacaagcttgcggccgcactcgagcacca

SEQ ID NO: 11 - DNA, 52 bp, synthetic construct
  source 1..52: mol_type = other DNA
  misc_feature 1..52: note = Portion of pE-SUMO Kan plasmid sequence
tcctaggcttaagctcgaggcagctgttcgaacgccggcgtgagctcgtggt

SEQ ID NO: 12 - protein, 252 aa, synthetic construct
  REGION 1..252: note = DAF 1-4
DCGLPPDVPNAQPALEGRTSFPEDTVITYKCEESFVKIPGEKDSVICLKGSQWSDIEEFCQRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGK